(19) United States
(12) Patent Application Publication
(10) Pub. No.: US 2006/0014214 A1
(43) Pub. Date: Jan. 19, 2006

(54) Urine preservation system

(75) Inventor: Tony Baker, Sonora, CA (US)

Correspondence Address: CATALYST LAW GROUP, APC, 9710 SCRANTON ROAD, SUITE S-170, SAN DIEGO, CA, 92121, US

(73) Assignee: Sierra Diagnostics, LLC

(21) Appl. No.: 11/138,543

(22) Filed: May 25, 2005

Related U.S. Application Data
(60) Provisional application No. 60/574,529, filed on May 25, 2004.

(51) Int. Cl. G01N 33/53; C12N 9/99

(52) U.S. Cl. 435/7.1; 435/184

(57) ABSTRACT

An improved method of preserving a molecule in a bodily fluid comprises: (1) providing a preservative solution comprising: (a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and (b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M; and (2) adding the preservative solution to the bodily fluid, thus preserving the molecule. The molecule can be a protein or a small molecule, such as a steroid. The invention also encompasses preservative compositions suitable for preserving proteins or small molecules, and kits. Preservative compositions can further include at least one enzyme inactivating component selected from the group consisting of manganese chloride, sarkosyl, and sodium dodecyl sulfate in the range of up to about 5% molar concentration. Compositions and methods according to the present invention have many diagnostic and forensic uses, in addition to being suitable for preparing compositions usable by hunters for attracting animals.

### CROSS-REFERENCES

This invention claims priority from Provisional Application Ser. No. 60/574,529, filed May 25, 2004 by Tony Baker, entitled “Urine Preservation System.” The contents of this application are incorporated herein in their entirety by this reference.

### BACKGROUND OF THE INVENTION

This invention is directed to compositions and methods for the preservation of urine, particularly for the preservation of macromolecules such as nucleic acids and proteins, as well as small molecules, in urine in a condition in which they can be recognized by reagents that specifically recognize macromolecules in a sequence-specific or conformation-specific manner, or specifically recognize small molecules, for subsequent testing and analysis.

Modern testing and treatment procedures have successfully reduced the prevalence and severity of many infectious diseases. For example, sexually-transmitted disease (STD) clinics regularly screen and treat patients for such diseases as gonorrhea and Syphilis. It is now well-known to identify infectious agents such as gonococci by analyzing a DNA sample. A genetic transformation test (GTT), such as Gonostat™ (Sierra Diagnostics, Inc., Sonora, Calif.), can be used to detect gonococcal DNA in specimens taken from the urethra of men, and the cervix and anus of women, according to Jaffe H W, Kraus S J, Edwards T A, Zubrzycki L. Diagnosis of gonorrhea using a genetic transformation test on mailed clinical specimens, J Inf Dis 1982; 146:275-279. A similar finding was also published in Whittington W L, Miller M, Lewis J, Parker J, Biddle J, Kraus S. Evaluation of the genetic transformation test, Abstr Ann Meeting Am Soc Microbiol 1983; p. 315.

The GTT is a test for biologically active or native DNA. For example, the Gonostat(3) GTT can be used to detect DNA such as gonococcal DNA in urine specimens. The Gonostat™ assay uses a test strain, Neisseria gonorrheae, ATCC 31953. This test strain is a mutant that is unable to grow into visible colonies on chocolate agar at 37° C. in 5% CO₂. Gonococcal DNA extracted from clinical material can restore colony growth ability to this test strain. The Gonostat™ assay is discussed in Zubrzycki L, Weinberger S S, Laboratory diagnosis of gonorrhea by a simple transformation test with a temperature-sensitive mutant of Neisseria gonorrhoeae. Sex Transm Dis 1980; 7:183-187.

It is not always possible to immediately test a patient for the presence of such an infectious agent. For example, clinical laboratories are not readily found in many rural or underdeveloped areas. In such circumstances, it is necessary to transport patient test specimens to a laboratory for analysis. It is therefore desirable to preserve such specimens for subsequent analysis with a GTT or other testing procedure.

Urine specimens are frequently practical and convenient for use in diagnoses of an infection, such as gonorrhea. A urine specimen can be collected by a patient, therefore avoiding the invasion of privacy and discomfort accompanying collection of other specimens, such as blood specimens, urethral cultures, or cervical cultures. Collection of a urine specimen by the patient also reduces the work load of the staff in the clinic or office.

DNA culture results of urine from males are quite sensitive when the urine is cultured within two hours of collection. Such results can approach 92% to 94%, or even 100%, as described in Schachter J. Urine as a specimen for diagnosis of sexually transmitted diseases. Am J Med 1983; 75:93-97. However, the culture results of urine from females are not very reliable, even when cultured within two hours. According to Schachter, only 47% to 73% of female urine cultures are positive relative to the culture results of cervical and anal specimens. Furthermore, it is known that culture results from any anatomic site are not 100% sensitive. (See, for example, Johnson D W, Holmes K K, Kvale P A, Halverson C W, Hirsch W P. An evaluation of gonorrhea casefinding in the chronically infected male. Am J Epidemiol 1969; 90:438-448; Schmale J D, Martin J E, Domescik G. Observations on the culture diagnosis of gonorrhea in women. JAMA 1969; 210:213-314; Caldwell J G, Price E V, Pazin G J, Cornelius E C. Sensitivity and reproducibility of Thayer-Martin culture medium in diagnosing gonorrhea in women. Am J Gynecol 1971; 109:463-468; Kieth L, Moss W, Berger G S. Gonorrhea detection in a family planning clinic: A cost-benefit analysis of 2,000 triplicate cultures. Am J Obstet Gynecol 1975; 121:399-403; Luciano A A, Grubin L. Gonorrhea screening. JAMA 1980; 243:680-681; Goh B T, Varia K B, Ayliffe P F, Lim F K. Diagnosis of gonorrhea by gram-stained smears and cultures in men and women: Role of the urethral smear. Sex Trans Dis 1985; 12:135-139.

Currently, urine specimens must be tested quickly for the presence of naked gonococcal DNA. Naked DNA is intact double stranded DNA which is released from viable gonococci. Such naked DNA can be found in the urine of an infected patient. However, enzymes in urine rapidly destroy any DNA present in the specimen. The DNA is either denatured, broken into single strands or totally destroyed by the enzymatic activity. This destruction of the DNA can effectively inactivate the naked gonococcal DNA for purposes of testing.

In a test such as the GTT, inactivation beyond the limits of detection is determined by the inherent genetic needs for select gene sequences of the Gonostat mutant strain used in the Gonostat test. For example, the Gonostat transformation assay is a very sensitive measurement tool for nucleic acid protection. In the GTT, the Gonostat organism must have approximately 1 picogram of native DNA to transform. This amount is equal to the presence of approximately 30 gonorrhea bacteria in an inoculum. The average clinical infection has 10³-10⁵ such organisms.

The destruction of DNA by enzyme activity in a urine specimen increases with time. For example, naked gonococcal DNA in a urine specimen that is stored in excess of two hours is inactivated beyond the limits of detection of the GTT. As a result, the testing of urine specimens for DNA is very time-sensitive. For example, DNA-based tests such as the polymerase chain reaction (PCR), the ligase chain technology (LCₓ) test of Abbott Laboratories, Abbott Park, Ill., and the GTT all must be performed on a urine specimen within approximately two hours. FIG. 1 is a graph of DNA concentration in unpreserved urine according to the prior art, demonstrating DNA destruction over time. The gonococcal DNA concentrations of ten different types of urine specimens were tested using a GTT at hourly intervals, commencing one hour from time of inoculation. Approximately 200 transformants were counted at the one hour measurement. However, for all specimens, the number of transformants declined by more than 100% within one hour of this initial measurement. The number of transformants approached zero within the two hours of the initial measurement, FIG. 2 is a graph of eight day serial data on unpreserved urine according to the prior art, further illustrating DNA destruction in unpreserved samples. Approximately seven transformants were counted at the one day measurement. However, by the second day, testing indicated that the biologically active DNA in the unpreserved urine had been totally destroyed by enzyme activity.

Tests such as the GTT can also be used to detect DNA in such bodily fluids and excretions as blood, blood serum, amniotic fluid, spinal fluid, conjunctival fluid, salivary fluid, vaginal fluid, stool, seminal fluid, and sweat. FIG. 3 is a graph of DNA concentration in unpreserved serum according to the prior art, demonstrating DNA destruction over time. The gonococcal DNA concentrations of normal and abnormal serum of both male and female were tested at hourly intervals, commencing from the time of inoculation. Approximately 100 transformants were counted at the one hour measurement. However, for all specimens, the number of transformants declined by more than 100% within three hours of this initial measurement. The number of transformants approached zero within the eight hours of the initial measurement.

Another test that can be used to identify DNA in a bodily fluid specimen is the PCR test. PCR testing uses discrete nucleic acid sequences and therefore can be effective even in the absence of intact DNA. FIG. 4 is a graph of PCR detection of MOMP Chlamydia in unpreserved urine according to the prior art, demonstrating DNA destruction over time. In PCR testing of an unpreserved urine specimen, four PCR absorbances were observed one hour after the addition of the MOMP Chlamydia. However, the number of PCR absorbances declined 100%, to two, when tested at two hours, and to zero by the third hour. This testing indicates that, even though PCR testing doesn't require intact DNA, the enzymatic activity of urine rapidly destroys even discrete nucleic acid sequences 45 within approximately three hours.

Unfortunately, practical and effective techniques for preserving DNA in certain bodily fluids have not been readily available. For example, one method used to deactivate urine enzymes is heating. In an experiment, urine was heated for five minutes in a boiling water bath (100° C.) and then cooled. Naked DNA and DNA released from gonococcal cells that were subsequently added to this urine were not deactivated. This suggests that the deoxyribonuclease component in urine is a protein(s), as proteins are typically denatured by such high temperatures.

However, heating can denature DNA that is already present in the urine specimen, including gonococcal DNA, as well as the DNA of Haemophilus influenzae and Bacillus subtilis. Thus, heating is not an appropriate method for preserving a patient urine specimen to test for the presence of such DNA. This is particularly true if the sample happens to be acidic, as heating DNA in an acidic medium can cause depurination, a reaction in which the purine bases are cleaved from the sugar-phosphate backbone. If depurination occurs, recognition reactions which depend for their specificity on the base sequence of the DNA become impossible.

In other known DNA assay systems, it is known to add detergents or other chemicals to assist in the detection of DNA. For example, in the DNA assay system described in Virtanen M, Syvanen A C, Oram J, Sodurlund H, Ranki M. Cytomegalovirus in urine: Detection of viral DNA by sandwich hybridization. J Clin Microbiol. 1984; 20:1083-1088, sarkosyl was used to detect cytomegalovirus (CMV) in urine by hybridization. In Boom R, Sol C J A, Salimans M M M, Jansen C L, Wertheim-van Dillen P M E, van der Noordaa J. Rapid and simple method for purification of nucleic acids. J Clin Microbiol 1990; 28:495-503, guanidinium chloride in urine was used to purify nucleic acids as assayed by gel electrophoresis. Although the reason for their use in these studies was not stated, the chemicals inactivated the deoxyribonuclease activity in urine that would have interfered with those assay systems.

It would therefore be advantageous to provide a method and system for preserving DNA in a bodily fluid such as urine, blood, blood serum, amniotic fluid, spinal fluid, conjunctival fluid, salivary fluid, vaginal fluid, stool, seminal fluid, and sweat, such that the efficacy of the DNA assays, e.g., the PCR, LCₓ, and the GTT is optimized.

Similarly, it would also be advantageous to provide a method and system for preserving proteins in a bodily fluid. If the primary sequence and three-dimensional structure of proteins in the bodily fluid can be preserved, many specific assays, including immunoassays, ligand-receptor assays and enzyme assays, can be run. However, as emphasized above, proteins in such bodily fluids can be subject to rapid degradation. Such degradation can be carried by the ubiquitin system.

Additionally, it would be extremely advantageous to provide a method and system for preserving small molecules in a bodily fluid, particularly urine. Many small molecules are participants in specific reactions, such as immunological reactions, antibody-antigen reactions, and reactions with receptors. Preserving the small molecules in a bodily fluid, therefore, can serve a number of purposes, including diagnostic and forensic. For example, the small molecules could be assayed for the diagnosis of conditions associated with the presence or abnormal concentration of such a small molecule. The small molecules could also be assayed for forensic purposes, such as might be needed in the prosecution of rapes and other crimes of violence.

One of those purposes is the use of urine as an attractant for animals, particularly in hunting and for fish bait. The use of fresh urine, such as fresh boar urine, as an attractant for animals is well known. However, the use of fresh urine requires its collection from animals just before its use, which is frequently messy, disagreeable, and inconvenient.

Applicant believes, without intending to be bound by this theory, that the components responsible for the activity of fresh urine in attracting animals are pheromones. Such pheromones can be steroids, which can occur free in solution or complexed with proteins. It would be desirable to preserve urine in such a way that the activity of these pheromones is preserved.

Accordingly, there is a requirement for methods and compositions that provide improved preservation and stabilization of many components of bodily fluids, particularly proteins and small molecules. Such methods and compositions should be readily usable and require a minimum of attention by the user. Such methods and compositions should also be capable of preserving proteins and small molecules for a significant period of time, even without refrigeration.

### SUMMARY OF THE INVENTION

One aspect of the present invention that meets these needs is a method of preserving a molecule selected from the group consisting of a protein and a small molecule in a bodily fluid, comprising the steps of:

(1) providing a preservative solution comprising:
(a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M; and

(2) adding the preservative solution to the bodily fluid, thus preserving the molecule.

If the molecule is a protein, it can be selected from the group consisting of enzymes, antibodies, receptor proteins, regulatory proteins, membrane proteins, and structural proteins. Typically, the protein is protected from degradation from the ubiquitin system.

If the molecule is a small molecule, it can be a steroid, such as a steroid having pheromone activity. The steroid can be selected from the group consisting of androsterone, testosterone, tetrahydrogestrinone, dehydrochlortestosterone, metandienone, methyltestosterone, androlone, oxandrolone, oxymetholone, stanozolol, and their analogues, precursors, and metabolites.

Typically, the bodily fluid is selected from the group consisting of urine, blood, serum, plasma, amniotic fluid, cerebrospinal fluid, seminal fluid, vaginal fluid, stool, conjunctival fluid, salivary fluid, and sweat. More typically, the body fluid is urine.

The preservative composition can further include at least one enzyme inactivating component selected from the group consisting of manganese chloride, sarkosyl, and sodium dodecyl sulfate in the range of up to about 5% molar concentration.

Another aspect of the present invention is a preservative composition for preserving a molecule selected from a protein and a small molecule comprising:

(1) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and

(2) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

Yet another aspect of the invention is a kit comprising:

(1 ) the preservative composition of the present invention as described above;

(2) a vessel for collecting a biological fluid in which a protein or small molecule is to be preserved; and

(3) instructions for use.

Still another aspect of the invention is a composition comprising:

(1) animal urine; and

(2) the preservative composition of the present invention as described above, such that the animal urine contains a pheromone in sufficient quantity to act as an attractant to an animal of the same species as the animal from which the animal urine comes.

Similarly, another aspect of the invention is a method of preserving pheromone activity of an animal urine comprising the steps of:

(1) providing a fresh animal urine containing pheromone activity; and

(2) adding the fresh animal urine to the preservative composition of the present invention as described above to preserve the pheromone activity at a level such that the urine containing the preservative composition acts as an attractant to an animal of the same species as the animal from which the animal urine comes.

Another aspect of the invention is a preserved fluid comprising:

(1) a preservative composition for preserving a molecule selected from a protein and a small molecule comprising:
(a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M; and

(2) a bodily fluid from a human or non-human subject.

### BRIEF DESCRIPTION OF THE DRAWINGS

The following invention will become better understood with reference to the specification, appended claims, and accompanying drawings, where:

FIG. 1 is a graph of DNA concentration in unpreserved urine according to the prior art.

FIG. 2 is a graph of eight day serial data on unpreserved urine according to the prior art.

FIG. 3 is a graph of DNA concentration in unpreserved serum according to the prior art.

FIG. 4 is a graph of PCR detection of MOMP Chlamydia in unpreserved urine according to the prior art.

FIG. 5 is a bar graph of DNA concentration in preserved urine according to one aspect of the invention.

FIG. 6 is a graph of eight day serial data on preserved urine according to one aspect of the invention.

FIG. 7 is a graph comparing PCR results in unpreserved and preserved normal urine according to one aspect of the invention.

FIG. 8 is a graph of eight day serial data on preserved serum according to one aspect of the invention.

FIG. 9 is a graph of DNA concentration in preserved serum according to one aspect of the invention.

FIG. 10 is a flow chart of the method for preserving DNA according to one embodiment of one aspect of the invention.

FIG. 11 is a diagram of the system for preserving DNA according to one embodiment of one aspect of the invention.

FIG. 12 graphically illustrates a comparison of signal response in PCR assays wherein the DNA has been treated with a preservative according to one aspect of the invention, and one which has not.

FIG. 13 illustrates the efficacy of reagents of the present invention to enhance signal response of a branched DNA assay of blood plasma samples subjected to various storage conditions.

FIG. 14 illustrates the efficacy of reagents of the present invention to enhance signal response of a branched DNA assay of blood serum and plasma samples.

FIG. 15 is a graph showing the interference of methemoglobin on PCR absorbance in a PCR amplification assay on hepatitis B sequences MD03/06 in unprotected serum;

FIG. 16 is a graph showing the improvement in attenuating the interference of methemoglobin on PCR absorbance in a PCR amplification assay on hepatitis B sequences MD03/06 in serum which has been treated with a preservative according to one aspect of the invention.

FIG. 17 illustrates the synergistic effect provided by the components of the inventive reagents in protecting hepatitis B sequences in serum stored at room temperature and subsequently subjected to MD03/06 PCR detection.

FIGS. 18A-18G are graphs showing the absence of preservative effect on gonococcal DNA in urine stored at room temperature and subsequently subjected to PCR detection offered by the individual addition of certain components which are included in the reagents of the invention.

FIGS. 19A-19E are graphs showing comparisons of preservation of androsterone in androsterone-spiked human urine over 12 months: FIG. 19A: guanidinium HCl/EDTA versus potassium acid phosphate; FIG. 19B: guanidinium HCl/EDTA versus boric acid; FIG. 19C: guanidinium HCl/EDTA versus sodium bicarbonate; FIG. 19D: guanidinium HCl/EDTA versus benzoic acid; and FIG. 19E: guanidinium HCl/EDTA versus sodium benzoate.

FIG. 20 is a graph showing the prevention of degradation of protein AF176555 (calpain) in urine by the ubiquitin-28S proteasome pathway using single agents and combination agents; with chaotropic agents used at 2 M and chelators at 0.1 M. The single agents were sodium thiocyanate, guanidinium thiocyanate, guanidinium HCl, sodium perchlorate, and EDTA. The combination agents were sodium thiocyanate+EDTA, guanidinium thiocyanate+EDTA, guanidinium HCl+EDTA, sodium perchlorate+EDTA, and lithium chloride+EDTA.

FIG. 21 is a graph showing the survival of ubiquitin activating enzymes Ubc2 (E-2) and Ubc3 (E-2) in urine with and without 2M sodium thiocyanate and 0.1 M EDTA.

FIG. 22 is a graph showing the survival of protein AF068706 (G2AD) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein.

FIG. 23 is a graph showing the survival of Protein NM_{—}015416 (cervical cancer proto-oncogene protein p40) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein.

FIG. 24 is a graph showing the survival of ATP in urine with and without exposure to 2 M sodium thiocyanate+0.1 M EDTA.

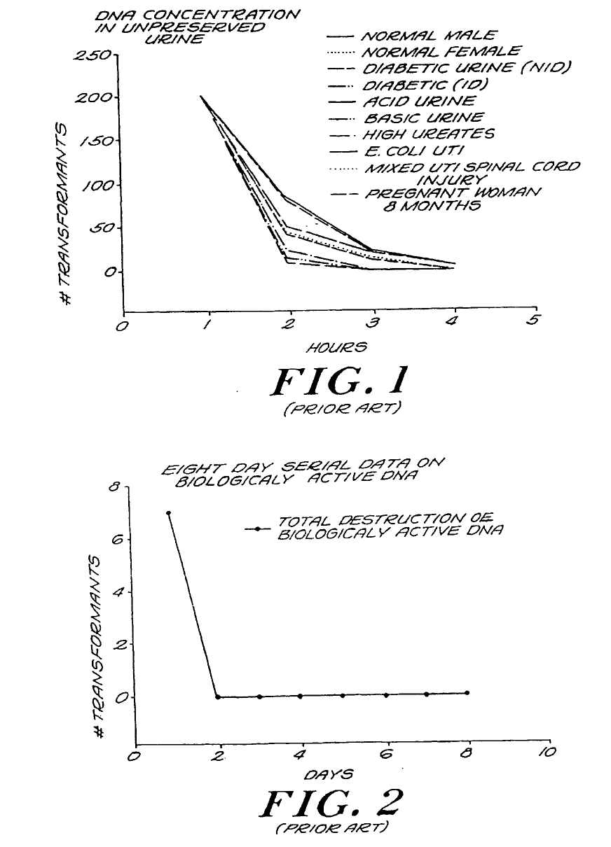

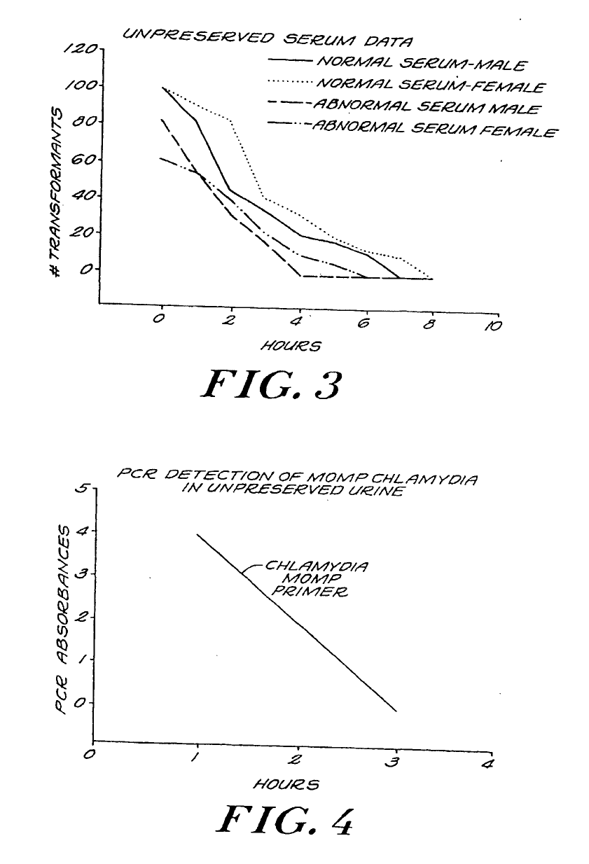

### DESCRIPTION OF THE PREFERRED EMBODIMENT

Improved methods, systems and reagents for preserving nucleic acids, e.g., DNA and RNA; proteins; and small molecules in bodily fluids are disclosed herein. The small molecules can be, but are not limited to compounds that can act as pheromones, such as steroids, either free or complexed with proteins. In one advantageous embodiment, the invention is may be used for preservation of nucleic acids, proteins, or small molecules such as steroids in urine. In another advantageous embodiment, the invention enables the molecular assay of nucleic acids, proteins, or small molecules in other bodily fluids and excretions, such as blood, blood serum, amniotic fluid, spinal fluid, conjunctival fluid, salivary fluid, vaginal fluid, stool, seminal fluid, and sweat to be carried out with greater sensitivity, as the methods and preservatives of the invention have been found to surprisingly increase the signal obtained with such nucleic acid testing methods as the polymerase chain reaction (PCR), LCₓ, and genetic transformation testing (GTT). In particular, the invention has also been found to surprisingly modulate the effect of hemoglobin, e.g., methemoglobin, interference on nucleic acid assays such as PCR on serum samples. Additionally, hybridization in such nucleic acid testing methods is unexpectedly improved. The specification of U.S. Pat. No. 6,458,546 to Baker is incorporated herein by this reference.

In an embodiment, the invention relates to methods of preserving a nucleic acid in a fluid such as a bodily fluid, including providing a nucleic acid preservative solution comprising an amount of a divalent metal chelator selected from ethylenediaminetetraacetic acid (EDTA), [ethylenebis(oxyethylenenitrilo)] tetraacetic acid (EGTA) and 1,2-bis(2-aminophenoxy)ethane-N,N,N′,N′-tetraacetic acid (BAPTA), and salts thereof; and an amount of at least one chelator enhancing component selected from lithium chloride, guanidine, sodium salicylate, sodium perchlorate, and sodium thiocyanate; and adding the nucleic acid preservative to the fluid, e.g., a bodily fluid. The amount of the divalent metal chelator is generally in the range of from about 0.001M to 0.1M, and the amount of the chelator enhancing component is generally in the range of from about 0.1M to 2M. The amount of chelator enhancing component is more desirably at least 1M in the preservative solution, and the divalent metal chelator is desirably present in an amount of at least about 0.01M.

In another embodiment, in which the invention relates to preserving a protein or a small molecule, such as a compound acting as a pheromone, the method includes providing a preservative solution comprising an amount of a divalent metal chelator selected from ethylenediaminetetraacetic acid (EDTA), [ethylenebis(oxyethylenenitrilo)]tetraacetic acid (EGTA) and 1,2-bis(2-aminophenoxy)ethane-N,N,N′,N′-tetraacetic acid (BAPTA), and salts thereof; and an amount of at least one chelator enhancing component selected from lithium chloride, guanidinium chloride, guanidinium thiocyanate, sodium salicylate, sodium perchlorate, and sodium thiocyanate; and adding the preservative solution to the fluid, e.g., a bodily fluid. The amount of the divalent metal chelator is generally in the range of from about 0.001 M to 2 M, and the amount of the chelator enhancing component is generally in the range of from about 0.1M to 10 M. The amount of chelator enhancing component is more desirably at least 1 M in the preservative solution, and the divalent metal chelator is desirably present in an amount of at least about 0.01 M, particularly when the preservation of proteins or small molecules is desired. The bodily fluid is typically urine, but can be another bodily fluid as described below. The bodily fluid can be a bodily fluid from a human subject, or a bodily fluid from a non-human animal, such as a socially or economically important animal such as a cow, a goat, a sheep, a pig, a dog, a horse, or a cat, or an animal that is hunted or tracked, such as a deer, a fox, a bear, a boar, an elk, a moose, or a raccoon. When the bodily fluid is human, the bodily fluid can have diagnostic or forensic applications as discussed below.

In this embodiment of the invention, in which the invention relates to preserving a protein or a small molecule, such as a compound acting as a pheromone, the amount of the divalent metal chelator can be increased so that it is in the range of from about 0.001 M to about 2 M. Similarly, the amount of the chelator enhancing component can be increased so that it is in the range of from about 0.1 M to about 10 M. These concentrations can be increased advantageously, because, when the invention relates to preserving a protein or a small molecule, it is typically unnecessary to use concentrations of divalent metal chelator and chelator enhancing component low enough so that there is substantially no interference with a nucleic-acid-hybridization-dependent assay such as PCR. As indicated above, he amount of chelator enhancing component is more desirably at least 1 M in the preservative solution, and the divalent metal chelator is desirably present in an amount of at least about 0.01 M, particularly when the preservation of proteins or small molecules is desired.

Accordingly, another aspect of the invention is a preservative composition for preserving a molecule selected from the group consisting of a protein and a small molecule comprising:

(1) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and

(2) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

As indicated above, the preservative composition can further comprise at least one enzyme inactivating component selected from the group consisting of manganese chloride, sarkosyl, and sodium dodecyl sulfate in the range of up to about 5% molar concentration.

As also indicated above, the amount of chelator enhancing component is more desirably at least 1 M in the preservative solution, and the divalent metal chelator is desirably present in an amount of at least about 0.01 M in the preservative solution, particularly when the preservation of proteins or small molecules is desired.

Additionally, when the method is used to preserve a protein, and it is subsequently desired to use the protein for a purpose, such as an immunoassay, in which the presence of high concentrations of divalent metal chelator or chelator enhancing component may be undesirable, the high concentrations of divalent metal chelator or chelator enhancing component can be removed by methods known in the art, such as equilibrium dialysis against a buffer containing lower concentrations of divalent metal chelator and chelator enhancing component or lacking these components. Another method is removal of the solvent by lyophilization followed by reconstitution in a desired buffer.

In another embodiment, the invention relates to preservative solutions comprising an amount of a divalent metal chelator selected from EDTA, EGTA and BAPTA, and salts thereof; and an amount of at least one chelator enhancing component selected from lithium chloride, guanidinium chloride, guanidinium thiocyanate, sodium salicylate, sodium perchlorate, and sodium thiocyanate. Preservative solutions according to the invention can be formulated to preserve nucleic acids, proteins, or small molecules such as steroids. When the preservative solution is formulated to preserve nucleic acids, the amount of the divalent metal chelator is generally in the range of from about 0.001 M to 0.1 M, and the amount of the chelator enhancing component is generally in the range of from about 0.1 M to 2 M. When the preservative solution is formulated to preserve nucleic acids, the amount of chelator enhancing component is more desirably at least 1 M in the preservative solution, and the divalent metal chelator is desirably present in an amount of at least about 0.01 M.

When the preservative solution is formulated to preserve proteins or small molecules, the amount of the divalent metal chelator is generally in the range from about 0.001 M to about 2 M, and the amount of the chelator enhancing component is generally in the range of from about 0.1 M to about 10 M.

The methods and preservatives of the invention can further include an amount of at least one enzyme inactivating component such as manganese chloride, sarkosyl, or sodium dodecyl sulfate, generally in the range of up to about 5% molar concentration.

In yet another aspect the invention relates to a method of improving the signal response of a molecular assay of a test sample, including providing a preservative solution comprising an amount of a divalent metal chelator selected from EDTA, EGTA and BAPTA, and salts thereof; and an amount of at least one chelator enhancing component selected from lithium chloride, guanidine, sodium salicylate, sodium perchlorate, and sodium thiocyanate; adding the preservative to a test sample to provide a preserved test sample; extracting molecular analytes of interest, e.g., DNA, RNA, proteins, or small molecules such as steroids from the preserved test sample, and conducting a molecular assay on the extracted molecular analytes of interest. The amount of the divalent metal chelator is generally as described above: e.g. in the range of from about 0.001 M to 0.1 M when the molecular analyte of interest is DNA or RNA, or in the range of from about 0.001 M to about 2 M when the molecular analyte of interest is a protein or a small molecule. Similarly, the amount of the chelator enhancing component is generally as described above: e.g. in the range of from about 0.1 M to 2 M when the molecular analyte of interest is DNA or RNA, or in the range of from about 0.1 M to about 10 M when the molecular analyte of interest is a protein or a small molecule. The chelator enhancing component is more advantageously one or more of sodium perchlorate, sodium thiocyanate, sodium perchlorate, guanidine, and lithium chloride. The amount of chelator enhancing component is more desirably at least 1 M in the preservative solution, and the divalent metal chelator is desirably present in an amount of at least about 0.01 M. When the molecular analyte of interest is DNA or RNA, signal response is believed to be enhanced in part due to enhanced hybridization as a result of the use of the reagents of the present invention.

In one aspect, when the methods and preservatives are used to preserve nucleic acids, use of the methods and preservatives disclosed herein eliminate enzymatic destruction of the nucleic acid of interest in the bodily fluid. The preservative can optionally be provided in solid or gaseous forms. While the methods and preservatives of the invention are useful in preserving all types of nucleic acids, e.g., RNA and DNA, including human DNA, and bacterial, fungal, and viral DNA, the invention is especially advantageous for use in preserving prokaryotic DNA, e.g., gonococcal DNA, DNA of Haemophilus influenzae and Bacillus subtilis. Nucleic acids in a bodily fluid are preserved for testing for a significantly longer period of time than that permitted by the prior art. While the maximum time between collecting, mailing, and testing patient specimens is expected to be approximately six days, the invention is effective beyond that period of time.

The preservatives of the invention may be used advantageously to preserve prokaryotic, e.g., gonococcal DNA, as shown below, although the teachings of the invention may be readily applied to the preservation of other types of DNA, including human, bacterial, fungal, and viral DNA, as well as to RNA. The reagents of the invention are believed to function by inactivating two classes of enzymes present in bodily fluids such as blood or urine which the inventor has recognized as destructive to DNA integrity, metal-dependent and metal independent enzymes. The divalent metal chelator removes, e.g., magnesium and calcium cation (Mg⁺², Ca⁺²) so as to effectively inactivate metal dependent enzymes such as deoxyribonucleases, a component of which has been found to inactivate gonococcal DNA in unpreserved urine. The divalent metal chelator may be ethylenediaminetetraacetic acid (EDTA), [ethylenebis(oxyethylenenitrilo)]tetraacetic acid (EGTA), or 1,2-bis(2-aminophenoxy)ethane-N,N,N′,N′-tetraacetic acid (BAPTA), or salts thereof. The amount of the divalent metal chelator is generally in the range of from about 0.001M to 0.1M when preservative solutions according to the present invention are used to preserve nucleic acids. More desirably, the amount of the divalent metal chelator in the preservative solution is at least 0.01M.

The second component of the reagents disclosed herein include a chelator enhancing component which assists the divalent metal chelator in protecting the nucleic acids in the fluid. These chelator enhancing components are believed to inactivate metal independent enzymes found in bodily fluids such as DNA ligases, e.g., D4 DNA ligase; DNA polymerases, e.g., T7 DNA polymerase; exonucleases, e.g., exonuclease 2, λ-exonuclease; kinases, e.g., T4 polynucleotide kinase; phosphatases, e.g., BAP and CIP phosphatase; nucleases, e.g., BL31 nuclease, and XO nuclease; and RNA-modifying enzymes such as E coli RNA polymerase, SP6, T7, T3 RNA polymerase, and T4 RNA ligase. Lithium chloride, guanidinium chloride, guanidinium thiocyanate, sodium salicylate, sodium perchlorate, and sodium thiocyanate have been found to be particularly effective. The amount of the chelator enhancing component is generally in the range of from about 0.1 M to 2 M when preservative solutions according to the present invention are used to preserve nucleic acids. More desirably the amount of chelator enhancing component in the preservative solution is at least 1 M.

The methods and preservatives of the invention have been found to surprisingly increase the signal obtained with such nucleic acid testing methods as the polymerase chain reaction (PCR), LCₓ, and genetic transformation testing (GTT). The invention has been found to surprisingly and unexpectedly enhance hybridization in such nucleic acid testing methods such as the PCR. FIG. 12 illustrates the improvement in hybridization obtained by use of a preservative disclosed herein on the hybridization of penicillinase-producing Neisseria gonorrheae (PPNG) DNA and PPNG-C probe.

A further aspect of the invention relates to methods of improving hybridization of nucleic acids, including contacting a test nucleic acid with a nucleic acid preservative solution comprising an amount of a divalent metal chelator selected from ethylenediaminetetraacetic acid (EDTA), ethylenebis(oxyethylenenitrilo)]tetraacetic acid (EGTA) and 1,2-bis(2-aminophenoxy)ethane-N,N,N′,N′-tetraacetic acid (BAPTA), or salts thereof in the range of from about 0.001 M to 0.1 M; and an amount of at least one chelator enhancing component selected from lithium chloride, guanidinium chloride, guanidinium thiocyanate, sodium salicylate, sodium perchlorate, and sodium thiocyanate in the range of from about 0.1 M to 2 M, such that a test solution is formed; and contacting the test solution with a target nucleic acid under conditions favorable for hybridization, such that hybridization occurs.

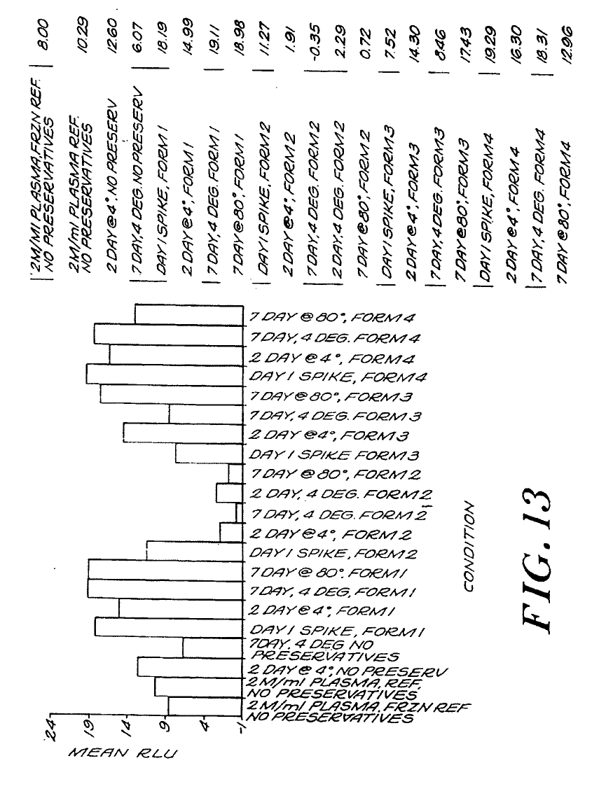

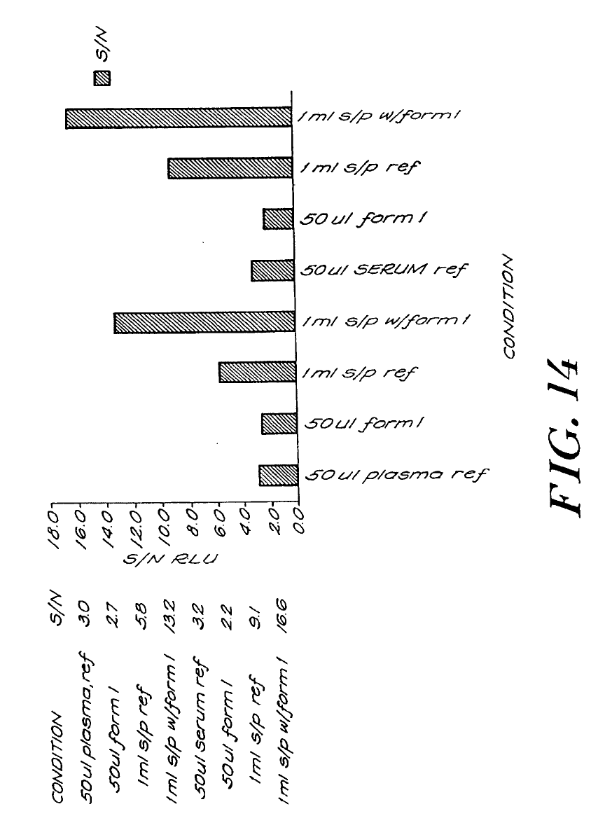

FIGS. 13 and 14 further illustrate the efficacy of the methods and preservatives of the invention in improving the results obtained with nucleic acid testing methods, in this case, a branched DNA (bDNA) assay (Chiron). In the tests run in FIG. 13, the bDNA assay was used to assess the protective effect of the DNA/RNA protect reagents. DNA sequences from the hepatitis C virus were spiked into serum and plasma. The protected serum and plasma were mixed with 9 ml of serum or plasma and 1 ml of preservative. The following formulations were used: 1) 1 M guanidine HCl/0.01 M EDTA, 2) 1 M sodium perchlorate/0.01 M BAPTA, 3) 1 M sodium thiocyanate/0.01 M EGTA, and 4) 1 M lithium chloride/0.01 M EGTA. The formulations were stored for seven days at 4° C. The bDNA assay relies on hybridization; it can clearly be seen from the absorbance results that the target sequences were not only protected against degradation, but the more than doubling of the absorbance results indicates an enhancement of hybridization/annealing of the target sequences.

FIG. 14 illustrates a serum versus plasma study in which 50 μl samples of fresh human plasma, and 1 ml samples of fresh human serum were protected with 1 M guanidine HCl/0.01 M EDTA and the bDNA assay was run on these samples after the samples were stored at 20° C. for 48 hours. Results were compared to unprotected samples. It can clearly be seen from the absorbance results that the target sequences were not only protected against degradation, but the more than doubling of the absorbance results indicates an enhancement of hybridization/annealing of the target sequences.

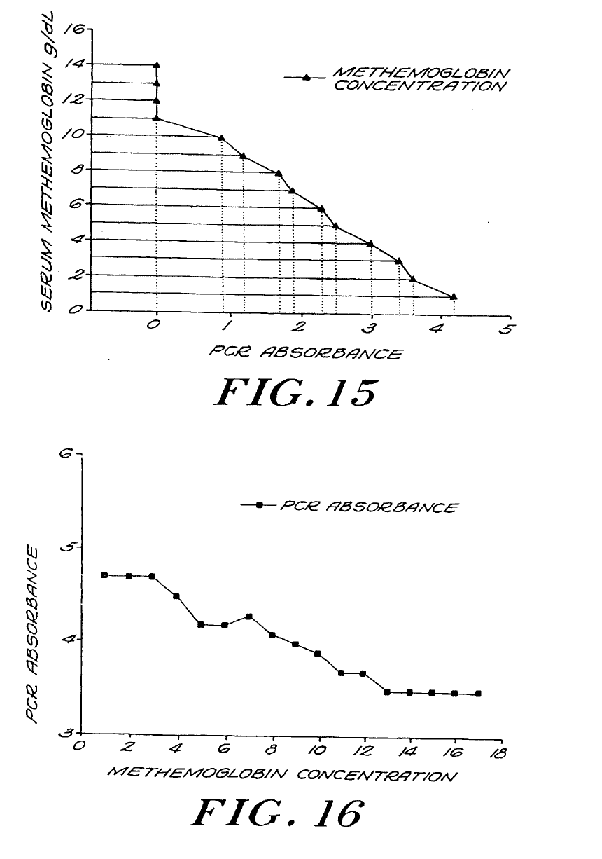

The preservative reagents of the invention have also surprisingly been found to remove the interference with heme compounds, e.g., methemoglobin, on PCR assays run on blood serum. FIGS. 15 and 16 illustrate the improvement obtained by use of the preservatives disclosed herein. Increasing amounts of methemoglobin were spiked into unprotected fresh human serum, to a concentration of 10 dl/ml. Serial PCR assays were run over a four hour period.

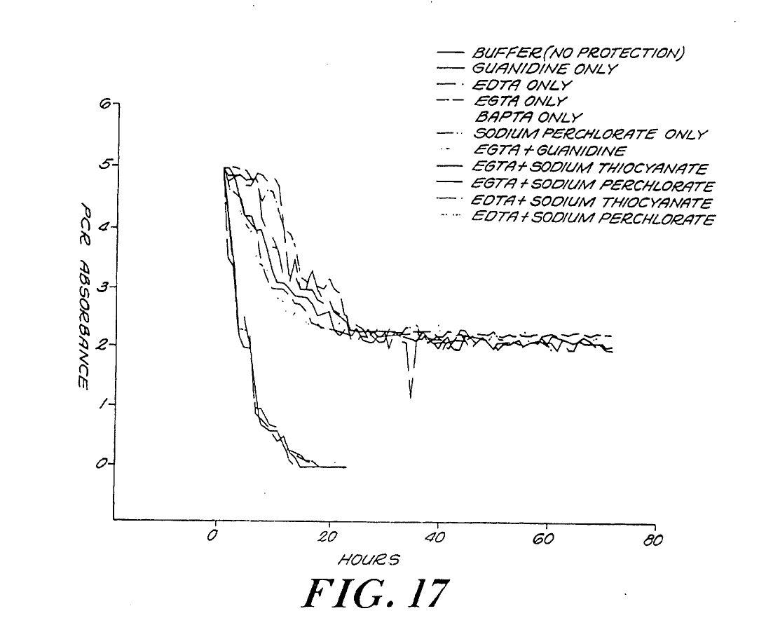

FIG. 17 illustrates the surprising and synergistic effect obtained by the combination of divalent metal chelators and chelator enhancing components in the inventive reagent (i.e., 1 M sodium perchlorate/0.01 M EGTA) in protecting hepatitis B sequences in serum stored at room temperature and subsequently subjected to MD03/06 PCR detection. The protocol run was as above (i.e., as illustrated in FIG. 16). It can be seen from the figures that compared to the addition of EGTA or sodium perchlorate individually, but protection of Hep B sequences is dramatically increased when preservative solutions of the present invention are used.

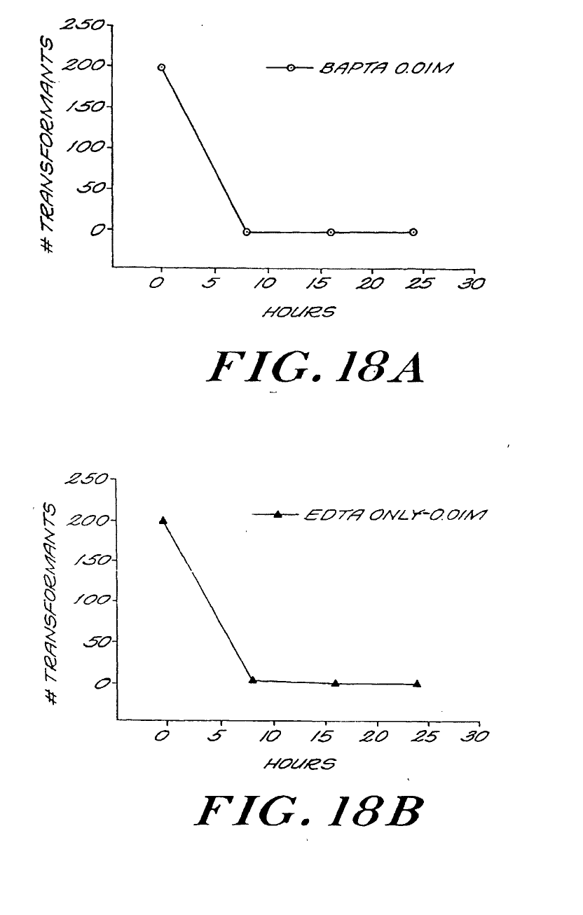

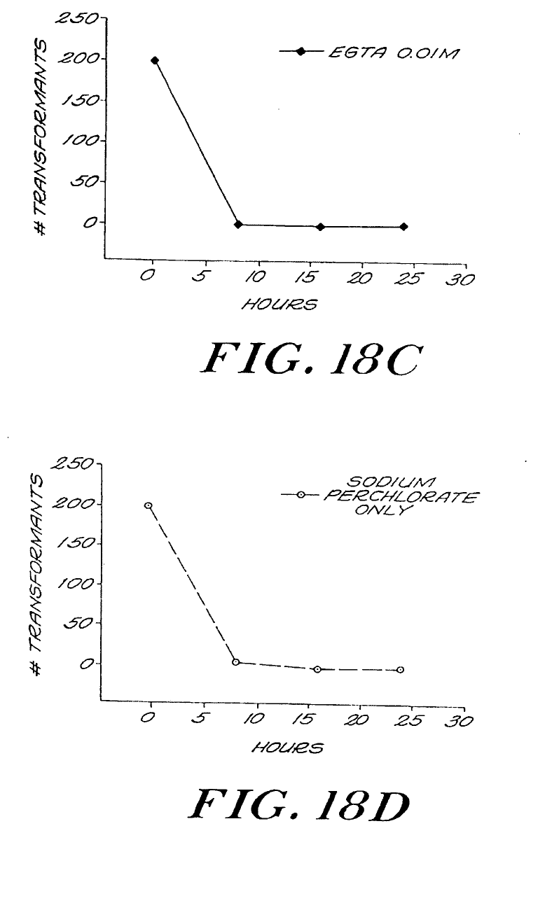

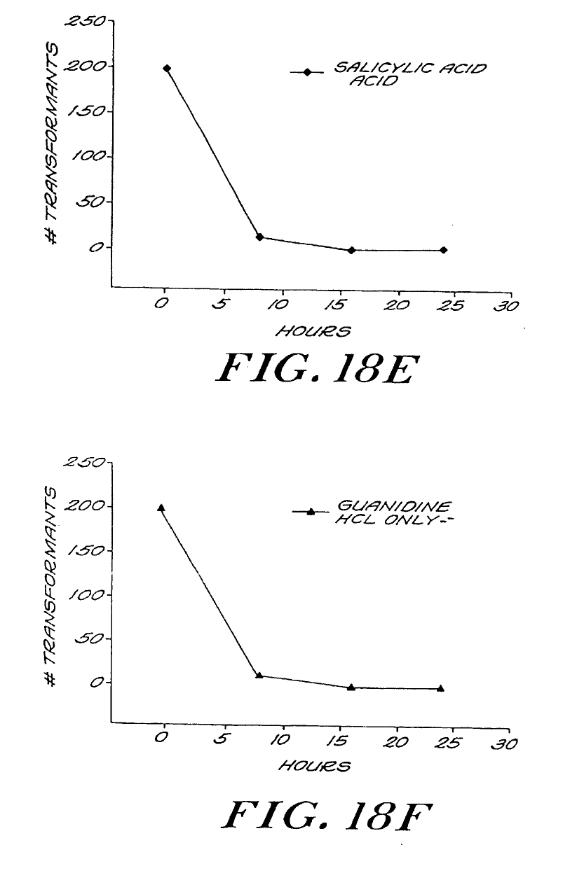

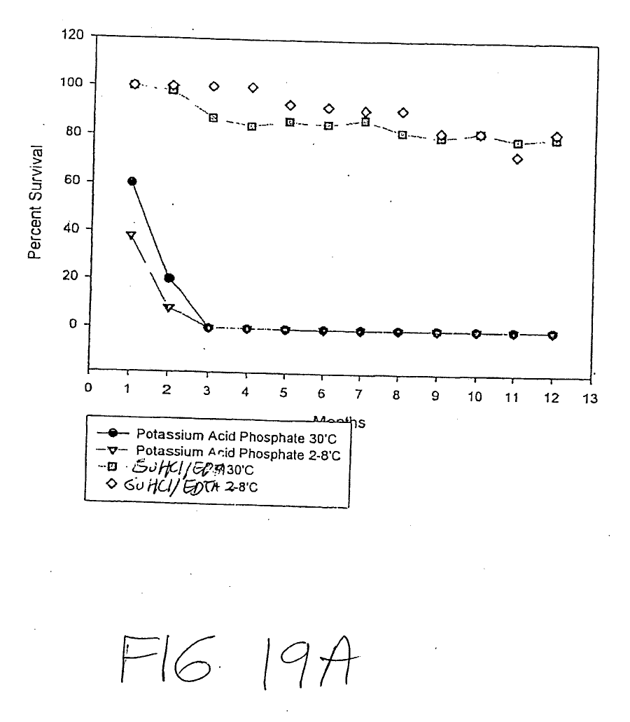

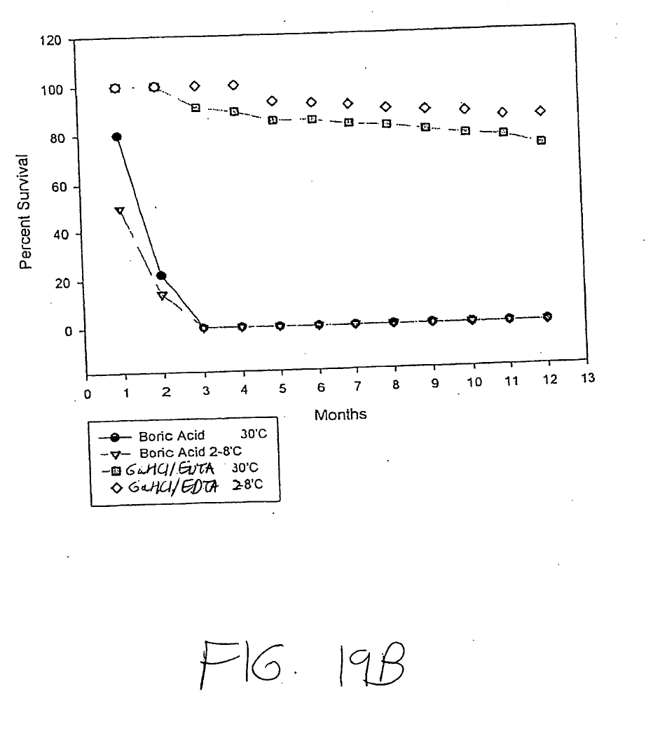

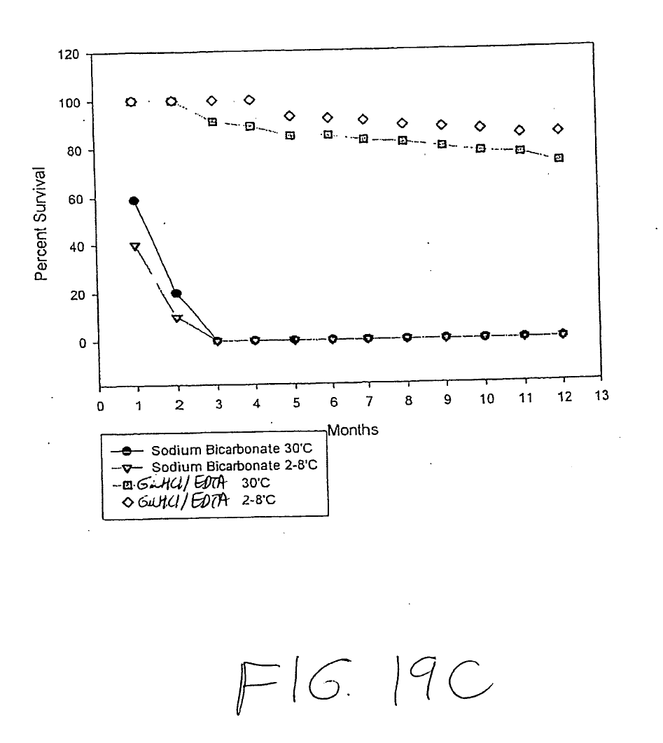

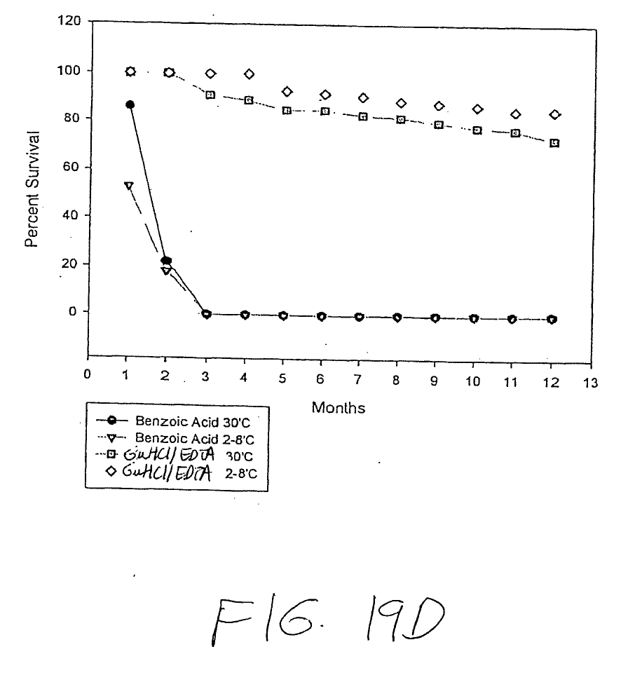

FIG. 18 illustrates the relatively weak preservative effect on gonococcal DNA in urine stored at room temperature and subsequently subjected to PCR detection offered by the individual addition of components of the reagents of the present invention, i.e., divalent metal chelators 0.01 M BAPTA (18A), 0.01 M EDTA (18B), 0.01 M EGTA (18C); and chelator enhancing components 1 M sodium perchlorate (18D), 1 M salicylic acid (18E), 1 M guanidine HCl (18F), 1 M sodium thiocyanate (18G), and 1 M lithium chloride (18H). The number of transformants in ten types of urine specimens were tested using a GTT, counted hourly, and then summarized. The standard Gonostat protocol (see Example 2, infra) was employed and illustrated the synergistic effect obtained by the combination of divalent metal chelators and chelator enhancing components in protecting gonococcal DNA in urine stored at room temperature and subsequently subjected to PCR detection.

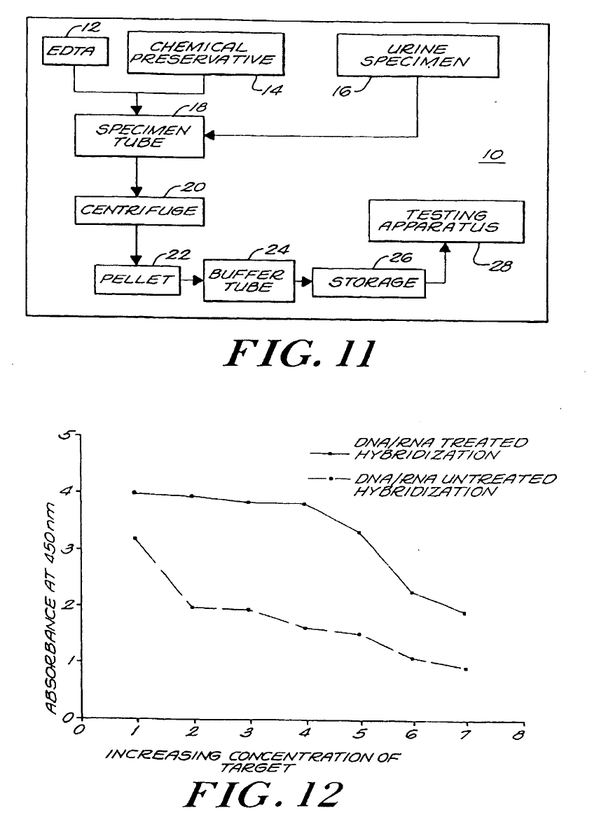

Another embodiment of the invention, a method 10 for preserving DNA, is illustrated diagrammatically in FIG. 11. This embodiment uses an exemplary protocol to preserve and test the urine specimens. The protocol is described in Table 1, below. This system produces high yields of DNA/RNA suitable for such testing methods as PCR, restriction fragment length polymorphisms assay (RFLP), and nucleic acid probes from urine specimens.

TABLE 1
1. 10 ml of clean catch urine 16 is added to a specimen test tube 18
containing divalent metal chelator 12 and chelator enhancing component
14. Test tube is inverted two or three times to mix the urine.
2. Test tube is transported to laboratory. No refrigeration is necessary.
Note: The test tube should be stored in a cool place and not in direct
sunlight.
3. At the laboratory, the test tube is centrifuged 20 at 3200 rpm for 10
minutes.
4. Using a sterile transfer pipette, the pellet 22 at the bottom of the test
tube is transferred to another test tube containing buffer 24. (As little
urine as possible should be transferred with the pellet material.)
5. The buffered material is stored 26 at between 2-8° C. until ready
to test 28.
6. The specimen size necessary to run the assay-needs to be validated on
the individual test methodology and individual testing protocol being used.

When the molecule to be preserved is a small molecule, it can be a steroid, such as a steroid with pheromone activity. An example of a steroid with pheromone activity is androsterone. The molecule to be preserved can also be another steroid, such as testosterone or a synthetic (“designer”) steroid such as tetrahydrogestrinone, dehydrochlortestosterone, metandienone, methyltestosterone, androlone, oxandrolone, oxymetholone, or stanozolol, as well as their analogues, precursors, and metabolites. With the increasing concern about the illegal and dangerous use of anabolic steroids among athletes, both amateur and professional, and the consequently increasing use of urine tests to detect such use, there is a need for a reliable method of preserving steroids in urine samples for later testing, supplied by methods and compositions according to the present invention.

When the molecule to be preserved is a protein, it can be a protein with any of a variety of biological activities, such as an enzyme, an antibody, a receptor protein, a regulatory protein, a membrane protein, or a structural protein. The protein can be monomeric or multimeric. If the protein is multimeric, methods and compositions according to the present invention are effective in preserving its quaternary structure; that is, the specific interaction between the subunits that is required to preserve the activity of the protein. In many cases, the protein is protected from degradation by way of the ubiquitin system.

When the molecule to be preserved is a protein, it can be a protein that is normally degraded by the ubiquitin system, degradation that catalyzed by activating enzymes E-1, E-2, E-3 in the presence of ATP and the 28S proteasome.

The biological fluid in which the nucleic acid, protein, or small molecule is to be preserved can be, but is not limited to, urine, blood, serum, plasma, amniotic fluid, cerebrospinal fluid, seminal fluid, vaginal fluid, stool, conjunctival fluid, salivary fluid, or sweat. Typically, the biological fluid is urine.

Accordingly, another aspect of the invention is a kit comprising: (1) a preservative composition according to the present invention; (2) a vessel for collecting a biological fluid in which a nucleic acid, protein, or small molecule is to be preserved; and (3) instructions for use. The vessel can contain the preservative composition ready for use; alternatively, the preservative composition can be packaged separately from the vessel. The preservative composition is as described above; when the molecule to be preserved is a protein or a small molecule, such as a steroid, higher concentrations of divalent metal chelator and chelator enhancing component can be employed.

Kits according to the present invention, as described above, can be used for testing or screening purposes. When such kits are used for testing or screening purposes, such kits can further comprise at least one sample containing the molecule to be preserved at a known concentration in the preservative composition. This sample can be used as a standard or a control in later testing, such as testing of human urine to determine the concentration of testosterone. The kit can include multiple samples containing the molecule to be preserved at a range of known concentrations, so that a standard curve can be run.

Another embodiment of the present invention is a composition comprising: (1) animal urine; and (2) a preservative composition of the present invention, such that the animal urine contains a pheromone in sufficient quantity to act as an attractant to an animal of the same species as the animal from which the animal urine comes. Typically, the animal urine is from an animal that is hunted, such as a deer (mule deer, whitetail deer, or other deer), a fox, a bear, a boar, an elk, a moose, or a raccoon. The pheromone can be a steroid, such as androsterone, but compositions of the invention are not limited to the preservation of steroids. In this embodiment of the invention, when a pheromone is preserved, higher concentrations of divalent metal chelator and chelator enhancing component are typically employed for maximum preservation of pheromone concentration.

Accordingly, another aspect of the invention is a method of preserving pheromone activity of an animal urine comprising the steps of: (1) providing a fresh animal urine containing pheromone activity; and (2) adding the fresh animal urine to a preservative composition of the present invention to preserve the pheromone activity at a level such that the urine containing the preservative composition acts as an attractant to an animal of the same species as the animal from which the animal urine comes.

The role of pheromones is described, for example., in B. Rasmussen, “Why Musth Elephants Use Pheromones,” Biologist 50: 195-196 (2003); R. Hudson, “Back to Basics: Expressive Behaviour,” at http://www.deer.rr.ualberta.ca/library/backtobasics/bbcommunication.htm; M. V. Novotny et al., “A Unique Urinary Constituent, 6-Hydroxy-6-Methyl-3-Heptanone, Is a Pheromone That Accelerates Puberty in Female Mice,” Chem. Biol. 6: 377-383 (1999); and “Pheromones: The Chemical Signals for Attraction,” at http://is2.dal.ca/˜kcollin2/pheromones.html, all of which are incorporated herein by this reference.

Yet another aspect of the invention is a preserved fluid comprising:

(1) a preservative composition for preserving a molecule selected from a protein and a small molecule comprising:
(a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M; and

(2) a bodily fluid from a human or non-human subject.

The preservative composition is as described above. The bodily fluid is typically urine, but can be another bodily fluid. As described above, the bodily fluid can have a human or non-human source.

The invention is illustrated by the following Examples. These Examples are included for illustrative purposes only, and are not intended to limit the invention.

### EXAMPLES

### Example 1

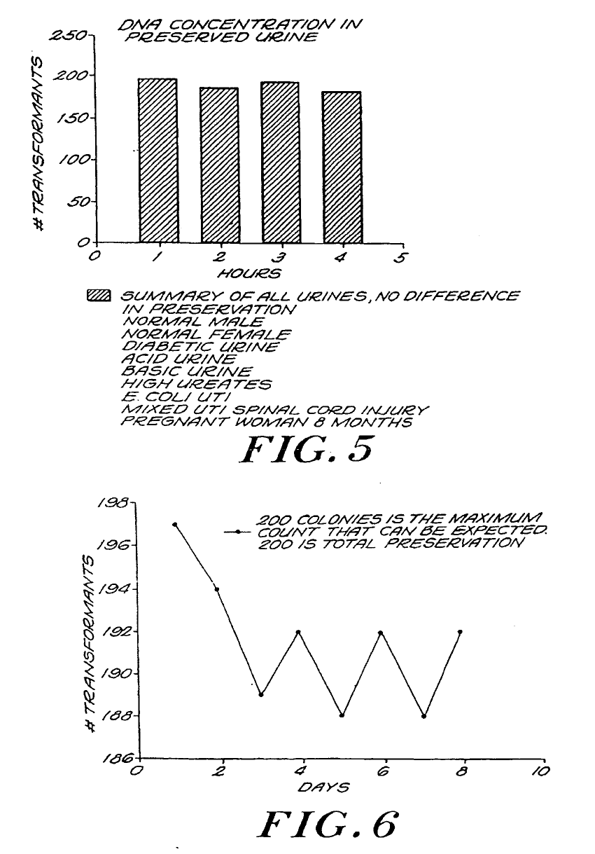

FIG. 5 is a bar graph of DNA concentration in preserved urine in accordance with the invention. The number of transformants in ten types of urine specimens were tested using a GTT, counted hourly, and then summarized. The standard Gonostat protocol (see Example 2, infra) was employed, and the preservative used was 1 M guanidine HCl/0.01 M EDTA. A count of two hundred colonies demonstrates total preservation of a specimen. The number of gonococcal transformants in the preserved urine remained relatively constant approaching two hundred, throughout the four hours of the test. No significant difference in level of preservation was observed among the different types of urine specimens. Therefore, it can be seen that the invention provides nearly total protection for DNA in urine.

### Example 2

FIG. 6 is a graph of eight day GTT serial data on preserved urine according to the invention. 1 pg of gonococcal DNA was spiked into 9 ml of fresh human urine and 1 ml of aqueous preservative containing 1 M sodium perchlorate and 0.01 M EGTA. 300 μl was spotted onto a lawn of the Gonostat organism at 24 hour intervals for eight days. The plates contained BBL Chocolate II agar and were incubated at 37° C. for 24 hours before readings were taken. The number of colonies observed throughout the eight-day testing period ranged from a low count of one hundred eighty-eight to a high count of one hundred ninety-seven. Thus, it can be seen that the invention preserves DNA in urine for a significantly longer period of time than previously provided.

### Example 3

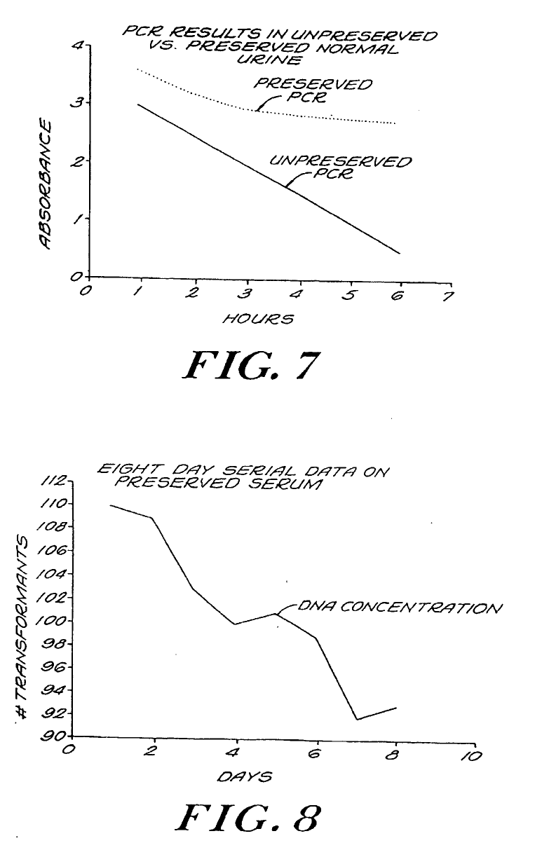

FIG. 7 is a graph comparing PCR results in unpreserved and preserved normal urine according to the invention. A MOMP template to Chlamydia trachomatis was used and amplified using a standard PCR protocol. 200 copies of the MOMP target were spiked into 9 ml of fresh human urine containing 1 M sodium perchlorate and 0.01 M BAPTA. PCR was done each hour for eight hours total. In the unprotected urine, approximately three PCR absorbances were measured one hour after the addition of DNA to the urine. The number of PCR absorbances approached zero by the sixth hour. By contrast, in the preserved specimen, in excess of three PCR absorbances were measured at the one hour testing. However, approximately three PCR absorbances were still observed by the sixth hour. Therefore, the invention preserves sufficient DNA and nucleic acid sequences to permit PCR testing well beyond the testing limits of unpreserved urine. The results shown in the Figure are consistent for all types of DNA in a urine specimen.

### Example 4

The reagents and methods of the invention may be used for preserving other bodily fluids and excretions, such as blood serum. FIG. 8 is a graph of eight day serial data on preserved serum according to the invention. The protocol used was similar to Example 3, except fresh human serum was used. The number of transformant colonies observed throughout the eight-day testing period ranged from a high count of one hundred ten at the one day measurement to a low count of approximately ninety-two at the seven day measurement. In fact, the test results actually showed an increase in transformant colonies between days seven and eight. Thus, it can be seen that the invention preserves DNA in serum for a significantly longer period of time than previously attainable.

### Example 5

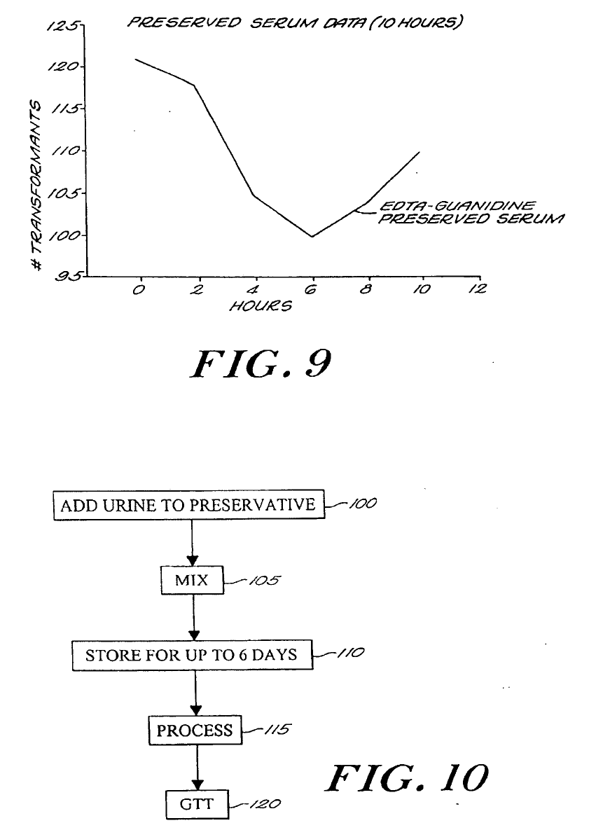

FIG. 9 is a graph of DNA concentration in preserved serum according to the invention. The serum was preserved with preservative solution comprising 1 M guanidine HCl/0.01 M EDTA. The protocol used was similar to Example 3, except fresh human serum was used, and the duration time of the study was ten hours. In excess of 120 transformants were measured at the time gonococcal DNA was added to the serum. Approximately 100 transformants were counted at the six hour measurement. However, by the tenth hour, testing indicated that the concentration of biologically active DNA in the preserved serum had increased to approximately 110 transformant colonies.

### Example 6

### Preservation of DNA in Simulated Clinical Specimens

In the following experiment, simulated clinical urine specimens were produced and tested for the presence of gonococcal DNA. The chemicals listed in Table 2, below, were added, at the concentrations previously described, to urine specimens from healthy adults, as was EDTA.

A suspension of gonococci was immediately added to each urine specimen. The added gonococci were an ordinary strain of N. gonorrheae, 49191, which was grown overnight on GC agar medium at 37° C. in a 5% CO₂ atmosphere. The N. gonorrheae colonies were picked and suspended in GC buffer. A 1/10 volume of a suspension containing approximately 10 Colony forming units (cfu) per ml was added to the urine. As a positive control, the suspension of gonococci was also added to Hepes buffer.

All simulated clinical specimens and the Hepes controls were tested at time zero, i.e., when the chemicals and gonococci were added. The specimens and controls were also tested after storage at room temperature for six days. This six day period was selected to approximate the maximum time expected between collecting, mailing, and testing patient specimens.

With the exception of urine samples containing SDS and sarkosyl, the simulated specimens and Hepes controls were processed as follows:

1. A 10 ml quantity was centrifuged at 4000 rpm for 30 minutes.

2. The supernatant was decanted, and the pellet was suspended in 1 ml phosphate buffer.

3. The suspension was heated for 10 minutes in a water bath at 60° C.

4. After cooling, the suspension was used in the GTT.

The simulated urine specimens containing SDS-EDTA or sarkosyl-EDTA were processed as follows:

1. Approximately a 2½ volume (approximately 25 ml) of 95% ethyl alcohol was added to the tube with the urine and preservative. The contents were mixed by inverting the tube several times.

2. The mixture was centrifuged at 4000 rpm for 30 minutes.

3. The pellet was suspended in 10 ml of 70% alcohol and centrifuged.

4. The pellet was then suspended in 1 ml phosphate buffer.

5. The suspension was heated for 10 minutes in a water bath at 60° C.

6. After cooling, the suspension was used in the GTT.

The inoculated urine was stored at room temperature for 6 days prior to testing. The formulations that preserved (+) or did not preserve (−) gonococcal DNA in the inoculated urine for six days to approximately the same degree as in the Hepes buffer control are indicated. Although the results of the Gonostat™ assay can be semi-quantitated, the tests were not designed to rank the relative efficacy of the chemical preservatives. Thus, the results given in Table 2 indicate whether or not the particular chemical preserved DNA in urine over a six day period to same degree as in the Hepes buffer.

TABLE 2
Preservative

Compositions Having Preservative Effect
0.01 M EDTA + 1 M Guanidinium Hydrochloride
0.01 M EDTA + 1 M Guanidinium Thiocyanate
0.01 M EDTA + 1 M Lithium Chloride
0.01 M EDTA + 1 M Manganese Chloride
0.01 M EDTA + 1% Sarkosyl
0.01 M EDTA + 1% Sodium Dodecyl Sulfate
0.01 M EDTA + 1 M Sodium Perchiorate
0.01 M EDTA + 1 M Sodium Salicylate
0.01 M EDTA + 1 M Sodium Thiocyanate
Compositions Having No Preservative Effect
1 M Sodium Periodate
1 M Trichioroacetic Acid
I M Urea

The 92% sensitivity exhibited with male urine specimens is comparable to the culture results reported in the literature. In addition, the 88% sensitivity exhibited with female urine specimens exceeds the previously-reported levels.

While a preferred embodiment of the invention is directed to the preservation of gonococcal DNA, it will be readily apparent to one skilled in the art that the invention is adaptable for use in preserving other types of DNA, such as that of Haemophilus influenzae and Bacillus subtilis. The invention can also be used to preserve RNA contained in bodily fluid samples. Such preserved RNA can be used for RNA transcriptase and reverse transcriptase assays for viral segments and human gene sequence testing. Additionally, the invention can be used to preserve proteins contained in bodily fluid samples, such as for immunological assays using suitable antibodies.

Furthermore, although in the preferred embodiment the preservatives are added to a bodily fluid, e.g., a urine specimen, the urine specimen can also be added to the preservatives without detriment to the efficacy of the invention. Optimal preservation of the DNA is typically and conveniently achieved by adding a single reagent of the invention to the specimen.

### Example 7

### PCR Detection of Penicillinase-producing Neisseria gonorrheae

The PCR signal-enhancing effect of the preservative reagents of the disclosure is demonstrated by the following example. Four varieties of TEM-encoding plasmids are found in PPNG. These are the 6.7 kb (4.4 Mda) Asian type, the 5.1 kb (3.2 Mda) African type, the 4.9 kb (3.05-Mda) Toronto type and the 4.8 kb (2.9-Mda) Rio Type. This PCR assay for PPNG takes advantage of the fact that the TEM-1 gene is located close to the end of the transposon Tn2; by the use of one primer in the TEM-1 gene and the other in a sequence beyond the end of Tn2, and common to all four plasmids, a PCR product only from plasmids and not from TEM-1 encoding plasmids was obtained. (Table 3, below) The conditions associated with this protocol were modified to include the DNA/RNA protect reagent in the hybridization and the treated probe was mixed with the 761-bp amplification product per standard PCR protocol. The results were read as absorbance at 450 nanometers.

Materials and Reagents
BBL chocolate 11 agar plates
Sterile Tris Buffer 10 mM Tris (pH 7.4), 1 mM EDTA
0.5-ml Gene Amp reaction tubes
Sterile disposable pasteur pipette tips
Aerosol-resistant tips
PCR master mix: 50 mM KCl, 2 mM MgCl₂, 50 μM each of deoxyribonucleoside
triphosphate; 2.5 U of taq Polymerase (Perkin Elmer); 5% glycerol; 50 pmol each of
primers PPNG-L and PNG-R (per 100 μl reaction)
Denaturation solution: 1 M Na 5×Denhardt×s solution
Prehybridization Solution:5×SSC(1×SSC is 0.015 M NaCl plus 0.015 M sodium citrate);
5×Denhardt's solution;
0.05% SDS;
0.1% sodium pyrophosphate, and
100 μg of sonicated salmon sperm DNA per ml.
Hybridization Solution
Same as prehybridization solution but without Denhardt's solution and including 200 μl of DNA/RNA protect reagent 1.
1 ml DNA/RNA preservative (1 M guanidine HCl/0.01 M EDTA)
Avidin-HRP peroxidase complex (Zymed)

Magnetic microparticles (Seradyne)

TABLE 3

| | | |
|---|---|---|
| Function | Name | Nucleotide Secuence 5′ to 3′ |

| | | |
|---|---|---|
| Primer | PPNG-L | AGT TAT CTA GAG GAG GG |
| | | (SEQ ID NO: 1) |
| Primer | PPNG-B | GGC GTA CTA TTC ACT CT |
| | | (SEQ ID NO: 2) |
| Probe | PPNG-C | GCG TCA GAG CCC TAT GTA TAA ACT C |
| | | (SEQ ID NO: 3) |

Methods

Sample preparation: 2 colonies were picked from a chocolate agar plate. Colonies were suspended in DI water just prior to setting up PCR. The master mix was prepared according to the recipe above. 5 μl of the freshly prepared bacterial suspension was added to 95 μl of master mix. The DNA was liberated and denatured in a thermocycler using three cycles of 3 min at 94° C. and 3 min at 55° C. The DNA was amplified in the thermal cycler by using a two step profile: a 25 s denaturation at 95° C. and a 25 s annealing at 55° C. for a total of thirty cycles. The time was set between the two temperature plateaus to enable the fastest possible annealing between the two temperatures. 15 pmol of labeled (avidin-HRP complex) detection probe PPNG-C was added to the hybridization solution bound to magnetic micro particles with and without the preservative reagent at 37° C. for 1 hour. The control and treated probes were then added to the amplification product and the reaction was colorimetrically detected by absorbance at 450 nm. The signal obtained from the hybridization probes treated with a reagent of the invention was found to be significantly higher than the untreated probes.

### Example 8

### Preservation of Androsterone in Human Urine

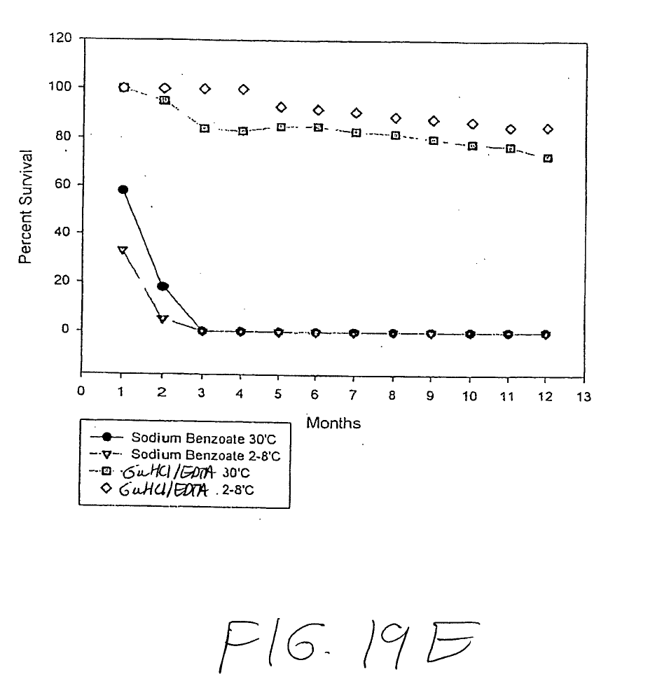

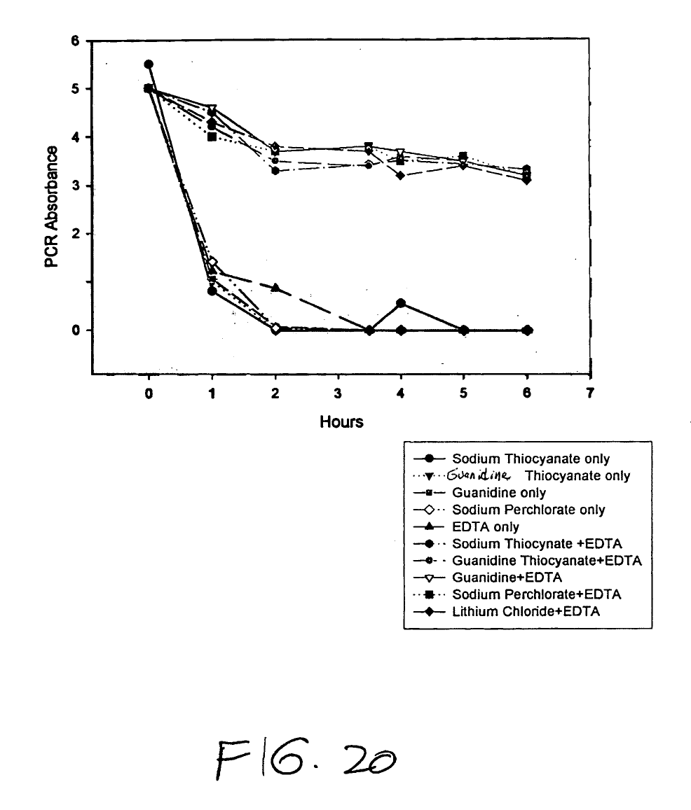

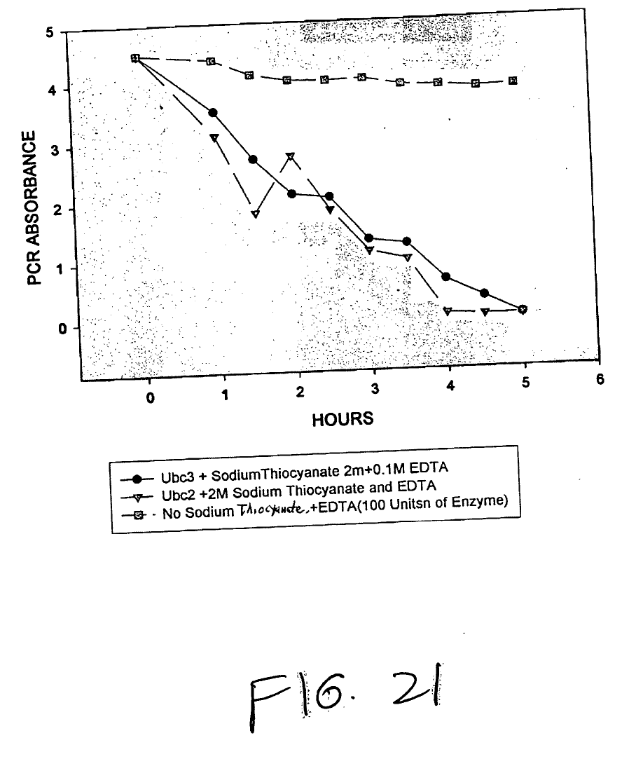

The formulation described above (1 M guanidinium HCl/0.01 M EDTA) was tested to determine its effectiveness in preserving the swine pheromone androsterone, a steroid, added to human urine. Human urine was used as a base, with the swine pheromone androsterone added to the solution. Solutions were prepared using the following preservatives: (1) 1 M guanidinium HCl/0.01 M EDTA; (2) potassium acid phosphate; (3) boric acid; (4) sodium bicarbonate; (5) benzoic acid; and (6) sodium benzoate. One portion of each of the six preservative solutions with the androsterone-spiked urine was kept at 8° C. and one portion was kept at 30° C. The solutions were maintained and tested monthly over a 12-month period. Testing was done by turbidity testing of antibody concentration using a spectrophotometer. The results are shown in the five comparison graphs as follows: FIG. 19A: guanidinium HCl/EDTA (“Gu/HCl/EDTA”) versus potassium acid phosphate; FIG. 19B: guanidinium HCl/EDTA versus boric acid; FIG. 19C: guanidinium HCl/EDTA versus sodium bicarbonate; FIG. 19D: guanidinium HCl/EDTA versus benzoic acid; and FIG. 19E: guanidinium HCl/EDTA versus sodium benzoate.

To summarize, the guanidinium HCl/EDTA solution preserved the androsterone molecules at or near the 100% level through four months, and over the next eight months maintained the androsterone levels at above 80% of the original concentration. Of the other preservatives, only one maintained androsterone concentration levels as high at 80% after even one month; none of the others maintained as much as a 20% concentration after two months, and all of the concentrations, other than the guanidinium HCl/EDTA test solution, were reduced to 0% by the third months.

Thus, the guanidinium HCl/EDTA solution preserved the steroid androsterone in urine over an extended period of time.

### Example 10

### Preservation of Proteins in Urine

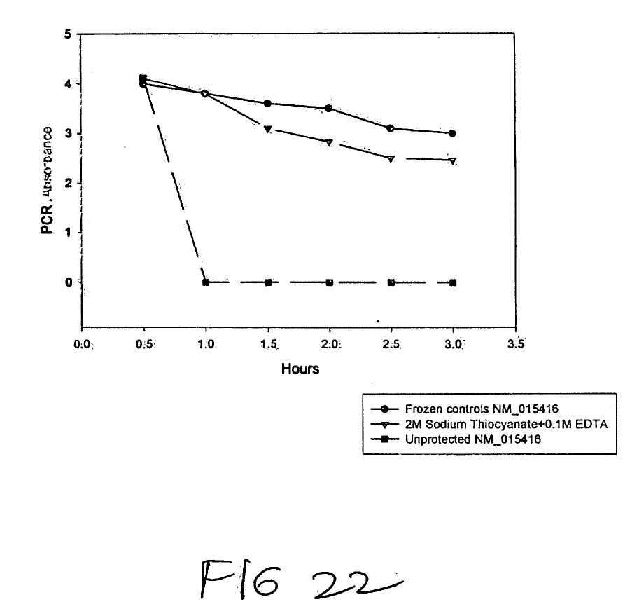

FIG. 20 shows the prevention of degradation of protein AF176555 (calpain) in urine by the ubiquitin-28S proteasome pathway using single agents and combination agents; with chaotropic agents used at 2 M and chelators at 0.1 M. The single agents were sodium thiocyanate, guanidinium thiocyanate, guanidinium HCl, sodium perchlorate, and EDTA. The combination agents were sodium thiocyanate+EDTA, guanidinium thiocyanate+EDTA, guanidinium HCl+EDTA, sodium perchlorate+EDTA, and lithium chloride+EDTA. The results shown in FIG. 20 show that the combination agents were substantially effective in preventing the degradation of calpain over 6 hours in urine; the single agents were substantially ineffective, with degradation occurring by 2 hours in most instances.

For the results in FIGS. 20-23, the proteins were quantitated by attaching appropriate PCR primers to segments of the protein so that PCR amplification would only occur on undegraded proteins, then performing PCR and quantitating the amount of amplification by absorbance. For the results in FIG. 24, the ATP was quantitated by immunoassay.

FIG. 21 shows the survival of ubiquitin activating enzymes Ubc2 (E-2) and Ubc3 (E-2) in urine with and without 2M sodium thiocyanate and 0.1 M EDTA. The ubiquitin-activating enzymes survived for a longer period of time without the sodium thiocyanate-EDTA. These results are consistent with protection of proteins that would normally be degraded by the ubiquitin system from degradation by the combination of sodium thiocyanate and EDTA.

FIG. 22 similarly shows the survival of protein AF068706 (G2AD) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein. The unprotected protein was degraded rapidly, while the protein protected with 2 M sodium thiocyanate and EDTA was protected nearly as well as frozen controls.

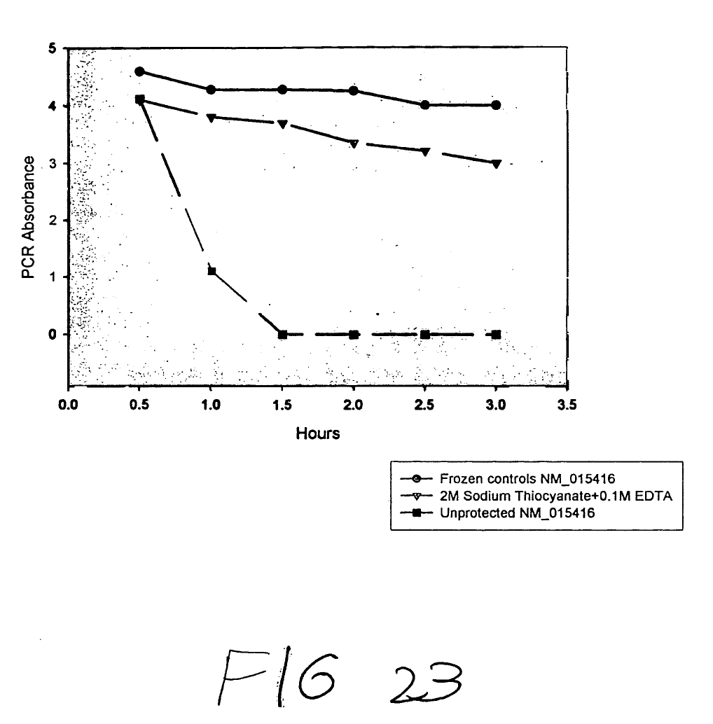

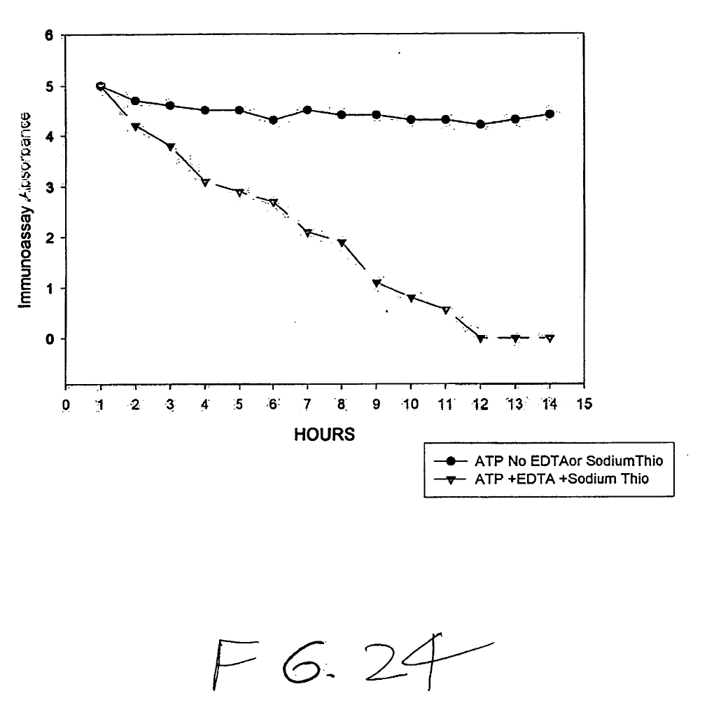

FIG. 23 similarly shows the survival of Protein NM_{—}015416 (cervical cancer proto-oncogene protein p40) from degradation by the ubiquitin system in urine spiked with ubiquitin, activating enzymes E-1, E-2, E-3, ATP, and 28S proteasome by 2 M sodium thiocyanate+0.1 M EDTA compared with frozen controls and unprotected protein. The unprotected protein was degraded rapidly, while the protein protected with 2 M sodium thiocyanate and EDTA was protected nearly as well as frozen controls.

FIG. 24 shows the survival of ATP in urine with and without exposure to 2 M sodium thiocyanate+0.1 M EDTA. ATP is degraded more rapidly in the presence of the 2 M thiocyanate and 0.1 M EDTA. Because ATP is involved in the degradation of proteins via the ubiquitin pathway, this result is consistent with the protection of proteins from degradation by the ubiquitin pathway by these reagents.

### ADVANTAGES OF THE INVENTION

The present invention provides compositions and methods that provide efficient preservation of nucleic acids, including DNA and RNA, proteins, including proteins subject to degradation by the ubiquitin system, and small molecules, including steroids, in bodily fluids. The proteins and small molecules are available for participation in specific reactions, including antigen-antibody reactions, enzymatic reactions, and receptor-binding reactions. These compositions and methods are useful in many applications, including diagnostic and forensic applications. They are also useful for providing a source of animal pheromones for hunters and fishermen.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. When a range of numerical values, such as concentrations, is recited in the specification and claims, such a range is deemed to include any possible value within the range unless specifically excluded. Therefore, a recitation of about 0.001 M to about 2 M is deemed to include, for example, 0.002 M, 0.003 M, and so on to the precision of measurement possible in the system. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

I claim:

1. A method of preserving a molecule selected from the group consisting of a protein and a small molecule in a bodily fluid, comprising the steps of:
(a) providing a preservative solution comprising:
(i) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(ii) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M; and
(b) adding the preservative solution to the bodily fluid, thus preserving the molecule.

2. The method of claim 1 wherein the molecule is a protein.

3. The method of claim 2 wherein the protein is selected from the group consisting of enzymes, antibodies, receptor proteins, regulatory proteins, membrane proteins, and structural proteins.

4. The method of claim 2 wherein the protein is protected from degradation by the ubiquitin system.

5. The method of claim 1 wherein the molecule is a small molecule.

6. The method of claim 5 wherein the small molecule is a steroid.

7. The method of claim 6 wherein the steroid is selected from the group consisting of androsterone, testosterone, tetrahydrogestrinone, dehydrochlortestosterone, metandienone, methyltestosterone, androlone, oxandrolone, oxymetholone, stanozolol, and their analogues, precursors, and metabolites.

8. The method of claim 1 wherein the bodily fluid is selected from the group consisting of urine, blood, serum, plasma, amniotic fluid, cerebrospinal fluid, seminal fluid, vaginal fluid, stool, conjunctival fluid, salivary fluid, and sweat.

9. The method of claim 1 wherein the bodily fluid is urine.

10. The method of claim 1 wherein the preservative composition further includes at least one enzyme inactivating component selected from the group consisting of manganese chloride, sarkosyl, and sodium dodecyl sulfate in the range of up to about 5% molar concentration.

11. The method of claim 1 wherein the concentration of divalent metal chelator is at least 0.01 M and the concentration of chelator enhancing component is at least 1.0 M in the preservative solution.

12. A preservative composition for preserving a molecule selected from a protein and a small molecule comprising:
(a) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(b) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M.

13. The preservative composition of claim 12 further comprising at least one enzyme inactivating component selected from the group consisting of manganese chloride, sarkosyl, and sodium dodecyl sulfate in the range of up to about 5% molar concentration.

14. The preservative composition of claim 12 wherein the molecule to be preserved is a protein.

15. The preservative composition of claim 12 wherein the molecule to be preserved is a small molecule.

16. The preservative composition of claim 15 wherein the molecule to be preserved has pheromone activity.

17. The preservative composition of claim 12 further comprising at least one enzyme inactivating component selected from the group consisting of manganese chloride, sarkosyl, and sodium dodecyl sulfate in the range of up to about 5% molar concentration.

18. The preservative composition of claim 12 wherein the concentration of divalent metal chelator is at least 0.01 M and the concentration of chelator enhancing component is at least 1.0 M.

19. A kit comprising:
(a) the preservative composition of claim 12;
(b) a vessel for collecting a biological fluid in which a protein or small molecule is to be preserved; and
(c) instructions for use.

20. The kit of claim 19 further comprising at least one sample containing the molecule to be preserved at a known concentration in the preservative composition.

21. A composition comprising:
(a) animal urine; and
(b) the preservative composition of claim 12, such that the animal urine contains a pheromone in sufficient quantity to act as an attractant to an animal of the same species as the animal from which the animal urine comes.

22. The composition of claim 21 wherein the animal urine is from an animal selected from the group consisting of a deer, a fox, a bear, a boar, an elk, a moose, and a raccoon.

23. The composition of claim 21 wherein the pheromone is a steroid.

24. A method of preserving pheromone activity of an animal urine comprising the steps of:
(a) providing a fresh animal urine containing pheromone activity; and
(b) adding the fresh animal urine to the preservative composition of claim 12 to preserve the pheromone activity at a level such that the urine containing the preservative composition acts as an attractant to an animal of the same species as the animal from which the animal urine comes.

25. A preserved fluid comprising:
(a) a preservative composition for preserving a molecule selected from a protein and a small molecule comprising:
(i) an amount of a divalent metal chelator selected from the group consisting of ethylenediaminetetraacetic acid (EDTA), (ethylenebis(oxyethylenenitrilo))tetraacetic acid (EGTA), and 1,2-bis(2-aminophenoxy)ethane-N,N,N′,N′-tetraacetic acid (BAPTA) and salts thereof in the range of from about 0.001 M to about 2 M; and
(ii) an amount of at least one chelator enhancing component selected from the group consisting of lithium chloride, guanidinium chloride, guanidinium thiocyanate, sodium salicylate, sodium perchlorate, and sodium thiocyanate in the range of from about 0.1 M to about 10 M; and
(b) a bodily fluid from a human or non-human subject.

26. The preserved fluid of claim 25 wherein the subject is human.

27. The preserved fluid of claim 28 wherein the bodily fluid is urine.

28. The preserved fluid of claim 25 wherein the preservative composition further includes at least one enzyme inactivating component selected from the group consisting of manganese chloride, sarkosyl, and sodium dodecyl sulfate in the range of up to about 5% molar concentration.

29. The preserved fluid of claim 25 wherein the concentration of divalent metal chelator is at least 0.01 M and the concentration of chelator enhancing component is at least 1.0 M in the preservative composition.


SEQUENCE LISTING (3 sequences)

SEQ ID NO: 1 - DNA, 17 bp, Artificial Sequence
  Artificial Sequence (primer)
agttatctacacgacgc

SEQ ID NO: 2 - DNA, 17 bp, Artificial Sequence
  Artificial Sequence (primer)
ggcgtactattcactct

SEQ ID NO: 3 - DNA, 25 bp, Artificial Sequence
  Artificial Sequence (primer)
gcgtcagacccctatctataaactc